(19) United States
(12) Patent Application Publication
(10) Pub. No.: US 2025/0100923 A1
(43) Pub. Date: Mar. 27, 2025

(54) GLASS CERAMIC AND MANUFACTURING METHOD THEREOF

(71) Applicant: NATIONAL SUN YAT-SEN UNIVERSITY, Kaohsiung City (TW)

(72) Inventors: YU-SHENG TSENG, Kaohsiung City (TW); WEN-FAN CHEN, Kaohsiung City (TW)

(21) Appl. No.: 18/530,283

(22) Filed: Dec. 6, 2023

(30) Foreign Application Priority Data
Sep. 21, 2023 (TW) 112136102

(51) Int. Cl. C03C 10/00; C03B 32/02

(52) U.S. Cl. CPC C03C 10/0009; C03B 32/02; C03C 4/0014; C03C 14/00

(57) ABSTRACT

A glass ceramic is provided to address the challenge that bioceramics known to be used for bone defect repair often lack good hardness, proper degradability, low post-implantation stimulation to surrounding tissues and promotion of bone defect repair in combination. The glass ceramic comprises a major crystallized phase, which is either diopside or wollastonite; and a minor crystallized phase, which comprises any one or more selected from the group consisting of diopside, wollastonite, lithium disilicate, silicon dioxide, lithium metasilicate and Li₂Ca₂Si₅O₁₃. In the glass ceramic, the molar ratio of elemental calcium, elemental lithium and elemental silicon is 1:x:2, in which x is from 0.05 to 1. However, when the major crystallized phase is diopside, the minor crystallized phase does not comprise diopside; and when the major crystallized phase is wollastonite, the minor crystallized phase does not comprise wollastonite. The present invention also comprises a method for manufacturing the glass ceramic.

### CROSS REFERENCE TO RELATED APPLICATION

The application claims the benefit of Taiwan application serial No. 112136102, filed on Sep. 21, 2023, and the entire contents of which are incorporated herein by reference.

### BACKGROUND OF THE INVENTION

### 1. Field of the Invention

The present invention relates to a ceramic and, more particularly, to a glass ceramic applicable to bone defect repair. The present invention further relates to a method for manufacturing the glass ceramic.

### 2. Description of the Related Art

In the field of bone defect repair, common materials for artificial bone substitutes and bone fixation devices available on the market include metal materials such as titanium alloy and stainless steel, and polymer materials like polylactic acid and polyglycolic acid.

Among them, metal materials such as titanium alloy and stainless steel are the most prevalent. However, these metal materials are non-degradable within the human body, potentially causing irritation to local tissues, abnormal temperature sensation at the implantation site, gradual deviation from the original position in the long run, and interference with radiographic examinations. Removal may necessitate a secondary surgical procedure.

Polymer materials such as polylactic acid and polyglycolic acid can undergo degradation within the human body. However, this degradation process generates acidic substances that may irritate surrounding tissues. Moreover, these polymer materials have relatively low mechanical strength, limiting their applicability to simple fracture repairs, and rendering them unsuitable for addressing complex fractures such as comminuted fractures.

In recent years, in the field of bone defect repair, implantable ceramics (i.e. bioceramics), have attracted significant attention due to their diverse biological activities that facilitate the promotion of bone regeneration.

Some studies have indicated that bioceramics based on wollastonite (CaSiO₃) exhibit excellent bone bonding characteristics. However, these bioceramics suffer from inadequate mechanical strength and an excessively rapid degradation rate, rendering them unfavorable for use as block implants. Moreover, during the initial stages of implantation, wollastonite-based bioceramics significantly impact the pH value of the surrounding environment, posing challenges to favorable conditions for cell growth.

In addition, bioceramics based on diopside (CaMgSi₂O₆) have excellent cell compatibility and, in comparison to wollastonite-based bioceramics, demonstrate superior mechanical strength. However, the incorporation of elemental magnesium reduces the formation of hydroxyapatite (referred to HAp hereinafter) on the surface of artificial implants produced with diopside-based bioceramics, thus affecting the attachment of bone cells.

In light of the above, it is necessary to improve the conventional bioceramics applied to bone defect repair.

### SUMMARY OF THE INVENTION

To address the above issues, the objective of the present invention is to provide a glass ceramic applicable to bone defect repair. The glass ceramic has both good hardness and proper degradability. Following its implantation into the human body, an artificial implant made of the glass ceramic induces minimal irritation to the surrounding tissues and promotes the bone defect repair.

Another objective of the present invention is to provide a method for manufacturing a glass ceramic, which is used to manufacture the aforementioned glass ceramic.

As used herein, the term “a”, “an” or “one” for describing the number of the elements and members of the present invention is used for convenience, providing the general meaning of the scope of the present invention, and should be interpreted to include one or at least one. Furthermore, unless explicitly indicated otherwise, the concept of a single component also includes the case of plural components.

The glass ceramic according to the present invention includes a major crystallized phase, which is either diopside or wollastonite; and a minor crystallized phase, which includes any one or more selected from the group consisting of diopside, wollastonite, lithium disilicate, silicon dioxide, lithium metasilicate and Li₂Ca₂Si₅O₁₃. In the glass ceramic, the molar ratio of elemental calcium, elemental lithium and elemental silicon is 1:x:2, in which x is 0.05 to 1. However, when the major crystallized phase is diopside, the minor crystallized phase does not comprise diopside; and when the major crystallized phase is wollastonite, the minor crystallized phase does not comprise wollastonite.

The glass ceramic according to the present invention is applicable to bone defect repair. For example, the glass ceramic can be prepared into an artificial implant for implantation into the human body. Due to the specific composition of the crystallized phase of the glass ceramic, the glass ceramic has the advantages of both wollastonite-based bioceramics and diopside-based bioceramics, and has not only good hardness, but also proper degradability. During bone defect repair, an artificial implant made of the glass ceramic can provide sufficient support while degrading gradually and slowly. Compared with wollastonite-based bioceramics, the glass ceramic will not greatly reduce the pH value in an environment surrounding the artificial implant during the degradation process, thereby reducing the irritation to the surrounding environment. Compared with diopside-based bioceramics, the glass ceramic can promote the formation of HAp on the surface of the artificial implant, so as to promote the attachment of bone cells. In short, the glass ceramic has a good bone repair effect, which is a key advantage of the present invention.

The glass ceramic may further include elemental magnesium, where the molar ratio of elemental lithium and elemental magnesium is x:(1-x), in which x is greater than or equal to 0.05 and less than 1. In this case, the glass ceramic has good hardness and proper degradability. When the glass ceramic is prepared into an artificial implant and implanted into the human body, no great impact is caused on the pH value in the environment surrounding the implantation site.

x may be 0.05 to 0.75. In this case, the glass ceramic has good hardness and proper degradability; and when the glass ceramic is prepared into an artificial implant and implanted into the human body, no great impact is caused on the pH value in the environment surrounding the implantation site.

x may be 0.25. In this case, the glass ceramic has good hardness and proper degradability; and when the glass ceramic is prepared into an artificial implant and implanted into the human body, no great impact is caused on the pH value in the environment surrounding the implantation site.

The method for manufacturing a glass ceramic according to the present invention is used to manufacture the aforementioned glass ceramic, and includes dissolving a glass ceramic raw material to obtain a solution of the glass ceramic raw material; adding a precipitant to the solution of the glass ceramic raw material to obtain a precipitate of the glass ceramic raw material; calcining the precipitate of the glass ceramic raw material to obtain a glass ceramic composition, wherein the molar ratio of elemental calcium, elemental lithium and elemental silicon is 1:x:2, in which x is 0.05 to 1; and sintering the glass ceramic composition.

By the method for manufacturing a glass ceramic according to the present invention, a glass ceramic with good bone repair effect as described above can be manufactured, which is a beneficial effect of the present invention.

The glass ceramic composition may further include elemental magnesium, where the molar ratio of elemental lithium and elemental magnesium is x:(1-x), in which x is greater than or equal to 0.05 and less than 1. In this case, the manufactured glass ceramic has good hardness and proper degradability; and when an artificial implant made of the glass ceramic is implanted into the human body, no great impact is caused on the pH value in the environment surrounding the implantation site.

The glass ceramic raw material may include: 17-23% by weight (wt %) of calcium chloride, 0.1-10 wt % of lithium chloride and 67-78 wt % of tetraethyl silicate. By using calcium chloride and lithium chloride as a calcium source and a lithium source in the method for manufacturing a glass ceramic according to the present invention, the occurrence of non-target crystallized phases produced after sintering can be reduced, thereby improving the quality of the glass ceramic. By using tetraethyl silicate as a silicon source in the method for manufacturing a glass ceramic according to the present invention, the toxicity of by-products is low, thereby improving the safety.

The glass ceramic raw material can further include less than or equal to 15 wt % and greater than 0 wt % of magnesium chloride. By using magnesium chloride as a magnesium source in the method for manufacturing a glass ceramic according to the present invention, the occurrence of non-target crystallized phases produced after sintering can be reduced, thereby improving the quality of the glass ceramic.

The calcining can be carried out at a temperature of 400-800° C. for 2-6 hrs thereby improving the quality of the glass ceramic.

The sintering can be carried out at a temperature of 900-1200° C. for 2-6 hrs, thereby improving the quality of the glass ceramic.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a flow chart of a method for manufacturing a glass ceramic of the present invention.

FIG. 2 shows X-ray diffraction patterns of pure diopside (Li0.00), as well as glass ceramics (Li0.25, Li0.50, Li0.75 and Li1.00).

FIG. 3 shows an SEM image of pure diopside (Li0.00) as a comparative example.

FIG. 4 shows an SEM image of a glass ceramic (Li0.25) of the present invention.

FIG. 5 shows an SEM image of a glass ceramic (Li0.50) of the present invention.

FIG. 6 shows an SEM image of a glass ceramic (Li0.75) of the present invention.

FIG. 7 shows an SEM image of a glass ceramic (Li1.00) of the present invention.

FIG. 8 shows an SEM image of pure diopside (Li0.00) as a comparative example after 28-day soaking in a simulated human body fluid.

FIG. 9 shows an SEM image of a glass ceramic (Li0.25) of the present invention after 28-day soaking in a simulated human body fluid.

FIG. 10 shows an SEM image of a glass ceramic (Li0.50) of the present invention after 28-day soaking in a simulated human body fluid.

FIG. 11 shows an SEM image of a glass ceramic (Li0.75) of the present invention after 28-day soaking in a simulated human body fluid.

FIG. 12 shows an SEM image of a glass ceramic (Li1.00) of the present invention after 28-day soaking in a simulated human body fluid.

FIG. 13 shows a histogram of hardness of pure diopside (Li0.00), as well as glass ceramics (Li0.25, Li0.50, Li0.75 and Li1.00), measured before soaking.

FIG. 14 shows a histogram of hardness of pure diopside (Li0.00), as well as glass ceramics (Li0.25, Li0.50, Li0.75 and Li1.00), measured after 28-day soaking in a simulated human body fluid.

FIG. 15 shows a line chart of weight loss of pure diopside (Li0.00), as well as glass ceramics (Li0.25, Li0.50, Li0.75 and Li1.00), measured after 28-day soaking in a simulated human body fluid.

FIG. 16 shows a line chart of pH changes over 28 days in simulated human body fluids in which pure diopside (Li0.00), as well as glass ceramics (Li0.25, Li0.50, Li0.75 and Li1.00), are immersed.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

### DETAILED DESCRIPTION OF THE INVENTION

To make the above and other objects, features and advantages of the present invention more obvious and comprehensible, preferred embodiments of the present invention will be described in detail with reference to accompanying drawings.

The glass ceramic according to the present invention can be understood to be manufactured by replacing elemental magnesium in the glass ceramic raw material used completely or partially with elemental lithium during the preparation of diopside (CaMgSi₂O₆) ceramic. In particular, 5-100% of elemental magnesium in the glass ceramic raw material is replaced by elemental lithium. As a result, in the manufactured glass ceramic, the molar ratio of elemental calcium:elemental lithium:elemental magnesium:elemental silicon is 1:x:(1-x):2, where x is 0.05 to 1.

The manufactured glass ceramic includes a major crystallized phase, which is either diopside or wollastonite (CaSiO₃). The so called “major crystallized phase” means the phase with the largest content in the composite crystallized phases of the glass ceramic. In particular, if elemental lithium is doped to replace elemental magnesium in the preparation process of diopside ceramics, a non-diopside crystallized phase will be formed. When the replacement ratio of elemental lithium is low (for example, when 25% of elemental magnesium in the glass ceramic raw material is replaced by elemental lithium), the major crystallized phase is still diopside. When the replacement ratio of elemental lithium is high (for example, when 75% of elemental magnesium in the glass ceramic raw material is replaced by elemental lithium), the major crystallized phase is wollastonite.

The manufactured glass ceramic further includes a minor crystallized phase, which includes any one or more selected from the group consisting of diopside, wollastonite, lithium disilicate (Li₂Si₂O₅), silicon dioxide (SiO₂), lithium metasilicate (Li₂SiO₃) and Li₂Ca₂Si₅O₁₃. The so-called “minor crystallized phase” means a phase in the composite crystallized phases of the glass ceramic that is less than the major crystallized phase. In particular, if elemental lithium is doped to replace elemental magnesium in the preparation process of diopside ceramics, wollastonite, lithium disilicate, silicon dioxide, lithium metasilicate, Li₂Ca₂Si₅O₁₃ and other crystallized phases are gradually formed, with increasing replacement ratio of elemental lithium. Notably, when the replacement ratio of elemental lithium is low such that diopside is still the major crystallized phase, the minor crystallized phase does not include diopside and includes wollastonite. As the replacement ratio of elemental lithium increases gradually, wollastonite will gradually replace diopside to serve as the major crystallized phase. In this case, the minor crystallized phase does not include wollastonite, but includes diopside. When elemental magnesium is completely replaced by elemental lithium, the crystallized phase of diopside disappears completely, and both the major and minor crystallized phases do not include diopside. For example, when 25% of elemental magnesium in the glass ceramic raw material is replaced by elemental lithium, diopside is the major crystallized phase, and wollastonite, lithium disilicate, silicon dioxide and others serve as the minor crystallized phase. When 75% of elemental magnesium in the glass ceramic raw material is replaced by elemental lithium, wollastonite is the major crystallized phase, and diopside, lithium disilicate, silicon dioxide, lithium metasilicate, Li₂Ca₂Si₅O₁₃ and others serve as the minor crystallized phase.

The manufactured glass ceramic further includes an amorphous phase (i.e. glass phase). In particular, the glass ceramic of the present invention includes more than or equal to 84.56% by weight (wt %) and less than 100 wt % of the crystallized phase, and greater than 0% and less than or equal to 15.44 wt % of the amorphous phase.

In the glass ceramic according to the present invention, the molar ratio of elemental calcium:elemental lithium:elemental magnesium:elemental silicon is 1:x:(1-x):2, where x is 0.05 to 1. In an embodiment, x may be 0.05 to 0.75, or 0.05 to 0.50, particularly 0.25. That is, 5-75% of elemental magnesium can be replaced by elemental lithium, or 5-50% of elemental magnesium can be replaced by elemental lithium, and particularly, 25% of elemental magnesium is replaced by elemental lithium.

By setting x to the above specific range, the glass ceramic of the present invention can have good hardness. Specifically, the Vickers hardness of the glass ceramic can be 300-700 Hv.

By setting x to the above specific range, the glass ceramic of the present invention can have proper degradability. Specifically, after the glass ceramic is immersed in a simulated human body fluid (pH 7.4) at 37° C. for 28 days, the weight loss rate can be from 0% to 5%.

By setting x to the above specific range, the artificial implant made of the glass ceramic has less impact on the pH value of the surrounding tissue after being implanted into the human body. Specifically, after the glass ceramic is immersed in a simulated human body fluid (pH 7.4) at 37° C. for 28 days, the pH of the simulated human body fluid is just increased to 8.0-8.4.

By setting x to the above specific range, the artificial implant made of the glass ceramic can promote the formation of HAp on the surface of the artificial implant after being implanted into the human body. Specifically, after the glass ceramic is immersed in a simulated human body fluid (pH 7.4) at 37° C. for 28 days, spherical HAp precipitate can be formed on the surface of the glass ceramic, where the diameter of the spherical HAp precipitate is about 5-12 μm.

Where the above molar ratio is met, the glass ceramic according to the present invention can be prepared by any known method, for example, coprecipitation, melting or solid-state reaction, etc. In an embodiment, the glass ceramic of the present invention was manufactured by coprecipitation. Compared with the melting or solid-state reaction using an oxide (such as calcium oxide, lithium oxide and silicon dioxide) or a carbonate as the raw material, coprecipitation has the advantages of simple operation, simple process, fine and uniform particle size, evenly distributed chemical elements, reduced cost, low energy consumption, and applicability to large-scale production.

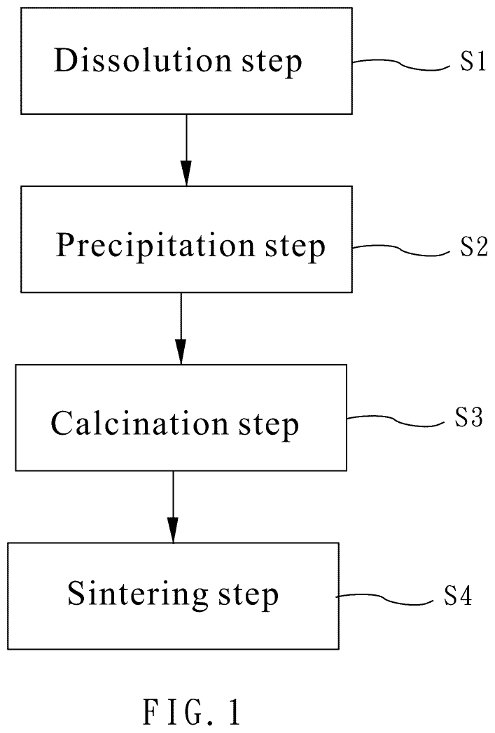

For example, referring to FIG. 1, a method for manufacturing the glass ceramic as described above includes: a dissolution step S1, a precipitation step S2, a calcination step S3 and a sintering step S4.

In particular, in the dissolution step S1, the glass ceramic raw material containing at least a calcium source, a lithium source and a silicon source is obtained firstly to provide the elemental calcium, elemental lithium and elemental silicon in the subsequently obtained glass ceramic composition. Then, the glass ceramic raw material is dissolved to obtain a solution of the glass ceramic raw material. For example, the calcium source can be calcium chloride (CaCl₂)) or calcium nitrate (Ca(NO₃)₂), the lithium source can be lithium chloride (LiCl) or lithium nitrate (LiNO₃), and the silicon source can be tetraethyl silicate (Si(OC₂H₅)₄) or tetramethyl silicate (Si(OCH₃)₄). Preferably, the glass ceramic raw material can contain, based on the total moles of elemental calcium, elemental lithium and elemental silicon in the glass ceramic raw material, 25-33 mol % of elemental calcium, 1-25 mol % of elemental lithium and 50-66 mol % of elemental silicon. In an embodiment, The glass ceramic raw material may include 17-23% by weight (wt %) of calcium chloride (CaCl₂)), 0.1-10 wt % of lithium chloride (LiCl) and 67-78 wt % of tetraethyl silicate (Si(OC₂H₅)₄). By using calcium chloride and lithium chloride as a calcium source and a lithium source in the co-precipitation, the occurrence of non-target crystallized phases produced after sintering can be reduced. By using tetraethyl silicate as a silicon source in the co-precipitation, the toxicity of the by-product (namely ethanol) is low, compared with the use of tetramethyl silicate.

The glass ceramic raw material may further include a magnesium source to provide elemental magnesium in the glass ceramic composition obtained subsequently. For example, the magnesium source can be magnesium chloride (MgCl₂) or magnesium nitrate (Mg(NO₃)₂). Preferably, the glass ceramic raw material can further contain, based on the total moles of elemental calcium, elemental lithium, elemental silicon and elemental magnesium, 0-24 mol % of elemental magnesium. In an embodiment, the glass ceramic raw material can further include less than or equal to 15 wt % and greater than 0 wt % of magnesium chloride (MgCl₂). By using magnesium chloride as a magnesium source in co-precipitation, the occurrence of non-target crystallized phases produced after sintering can be reduced. The glass ceramic raw material can be dissolved in a solvent that can be, for example, any organic solvent or water, provided that the calcium source, the lithium source, the silicon source and the magnesium source in the glass ceramic raw material can be completely dissolved. For example, the organic solvent can be an alcohol or an aqueous solution containing the alcohol, for example, methanol, ethanol or ethylene glycol, etc. In an embodiment, the glass ceramic raw material is dissolved in a 95% aqueous ethanol solution to obtain a solution of the glass ceramic raw material.

In the precipitation step S2, a precipitant is added to the solution of glass ceramic raw material to obtain a precipitate of the glass ceramic raw material. The precipitant can be any inorganic compound, provided that it can react with the elemental calcium, elemental lithium, elemental silicon and elemental magnesium in the solution of glass ceramic raw material to form a precipitate. For example, the inorganic compound can be ammonium hydroxide (NH₄OH), sodium hydroxide (NaOH), ammonium bicarbonate (NH₄HCO₃), sodium carbonate (Na₂CO₃), an inorganic compound containing a sulfate (SO₄²⁻) or an inorganic compound containing an oxalate (C₂O₄²⁻), and so on. In an embodiment, the inorganic compound can be a 28% ammonium hydroxide (NH₄OH) aqueous solution (aqueous ammonia).

In the calcination step S3, the precipitate of the glass ceramic raw material is calcined to obtain a glass ceramic composition. The “calcination” here is a heat treatment used for removing impurities in the precipitate of glass ceramic raw material (for example, impurity particles mixed in the process of preparing the precipitate of glass ceramic raw material, impurities in the original raw material, or impurities falling from the air) to obtain a pure oxide powder. If the calcination procedure is performed before sintering, holes formed when impurities are burned can be reduced, thereby improving the density of the end product. The calcination step S3 can be carried out at a temperature of 400-800° C. for 2-6 hrs. In an embodiment, the calcination step S3 is carried out at 600° C. for 3 hrs.

It is to be noted that in the glass ceramic composition obtained by the calcination step S3, the molar ratio of elemental calcium:elemental lithium:elemental magnesium:elemental silicon is 1:x:(1-x):2, in which x is 0.05 to 1.

Next, in the sintering step S4, the glass ceramic composition is sintered. In this way, the glass ceramic as mentioned above can be obtained. The “sintering” here is a heat treatment for densifying a powder or a pressed powder. In the present invention, at the high temperature of the sintering process, the glass ceramic composition is crystallized, to form a specific crystallized phase of the present invention. At this time, a glassy amorphous phase is also formed, to greatly improve the density of the ceramic. The sintering step S4 can be carried out at a temperature of 900-1200° C. for 2-6 hrs. In an embodiment, the sintering step S4 is carried out at 1000° C. for 4 hrs.

The aforementioned glass ceramic (or the glass ceramic manufactured according to the aforementioned method) can be applied to the repair of bone defects. For example, the glass ceramic can be prepared into an artificial bone, a bone nail, a bone plate and other artificial implants. Therefore, after the artificial implant is implanted into the human body, because the glass ceramic has good hardness and proper degradability, the artificial implant made of the glass ceramic can provide sufficient support while degrading gradually and slowly during bone defect repair. The artificial implant not only avoids a significant decrease in the pH value in the surrounding environment during the degradation process, thereby minimizing irritation to the surrounding environment, but also facilitates the formation of HAp on the surface of the artificial implant. Thus, this promotes the attachment of bone cells and effectively contributes to the repair of bone defects.

To prove that the glass ceramic can be used as an artificial implant, the following glass ceramics, which are respectively referred to as Li0.25, Li0.50, Li0.75 and Li1.00, are respectively prepared as test examples, and pure diopside (Li0.00) is prepared as a comparative example. The crystallized phase, morphology and porosity are compared, and the hardness, degradability, impact on pH value of surrounding tissues, and effect on promoting HAp formation are described.

### (I) Preparation of Samples

### Comparative Example: Pure Diopside (Li0.00)

0.01 mol calcium chloride, 0.01 mol magnesium chloride and 0.02 mol tetraethyl silicate (TEOS) were used as reactants, dissolved in 200 ml of 95% ethanol, and stirred at 80° C. for 2 hrs. 20 ml of a 28% ammonium hydroxide aqueous solution was added to produce a white precipitate. Ethanol was evaporated off by stirring overnight. The white precipitate was dried in an oven at 80° C. for 24 hrs. After drying, the white precipitate was ground into a powder, and calcined at 600° C. for 3 hrs to remove the impurity. The calcined powder was pressed at a pressure of 75 MPa for 30 sec to prepare tablets (diameter 13 mm, height 1.8 mm). Then the tablets were sintered in air at 1000° C. for 4 hrs to obtain pure diopside (Li0.00).

### Test Example: Li0.25, Li0.50, Li0.75, Li1.00

25%, 50%, 75%; and 100% of the 0.01 mol magnesium chloride used in the comparative example were respectively replaced by lithium chloride. Except for this, the preparation process was the same as that in the comparative example. Li0.25, Li0.50, Li0.75 and Li1.00 glass ceramics (in other words, the reactants in Li0.25 glass ceramic include 0.01 mol calcium chloride, 0.0025 mol lithium chloride, 0.0075 mol magnesium chloride and 0.02 mol tetraethyl silicate) were obtained respectively.

### (II) Identification of Crystallized Phase

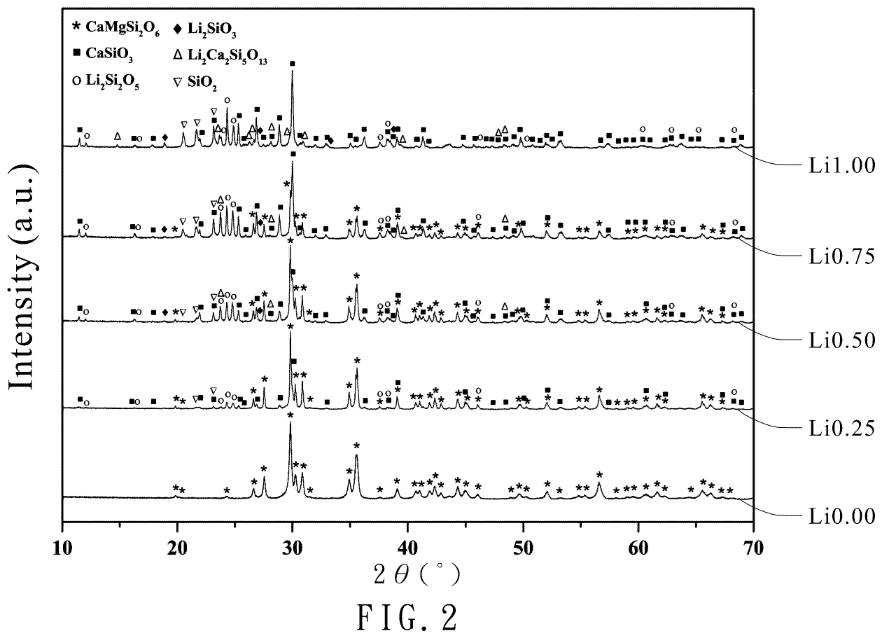

The sample was ground into a powder, and the crystallized phase was identified by X-ray diffraction. The results are shown in FIG. 2. The pure diopside sample (Li0.00) contains only diopside (CaMgSi₂O₆) crystallized phase (as indicated by “*”). When the replacement ratio of elemental lithium is increased to 25% (Li0.25), the formation of wollastonite (CaSiO₃, as indicated by “▪”), lithium disilicate (Li₂Si₂O₅, as indicated by “◯”), silicon dioxide (SiO₂, as indicated by “ [symbol] ”) and other minor crystallized phases can be detected. When the replacement ratio of elemental lithium is increased to 50% and 75% (Li0.50, Li0.75), the peak intensity of diopside (CaMgSi₂O₆) crystallized phase decreases, the peak intensities of wollastonite (CaSiO₃), lithium disilicate (Li₂Si₂O₅) and silicon dioxide (SiO₂) crystallized phases increase. Minor crystallized phases such as lithium metasilicate (Li₂SiO₃, as indicated by “♦”) and Li₂Ca₂Si₅O₁₃ (as indicated by “Δ”) are additionally formed. When the replacement ratio of elemental lithium is increased to 100% (Li1.00), the crystallized phase of diopside (CaMgSi₂O₆) disappears completely. The major crystallized phase is wollastonite (CaSiO₃), and the minor crystallized phase is lithium disilicate (Li₂Si₂O₅), silicon dioxide (SiO₂), lithium metasilicate (Li₂SiO₃) and Li₂Ca₂Si₅O₁₃.

Moreover, as can be seen from the X-ray diffraction pattern, with the increase of the replacement ratio of elemental lithium, the proportion of amorphous phase also increases. For example, the amorphous phase of Li0.25 is 4.46%, the amorphous phase of Li0.50 is 8.37%, the amorphous phase of Li0.75 is 12.26%, and the amorphous phase of Li1.00 is 15.44%.

### (III) Morphology

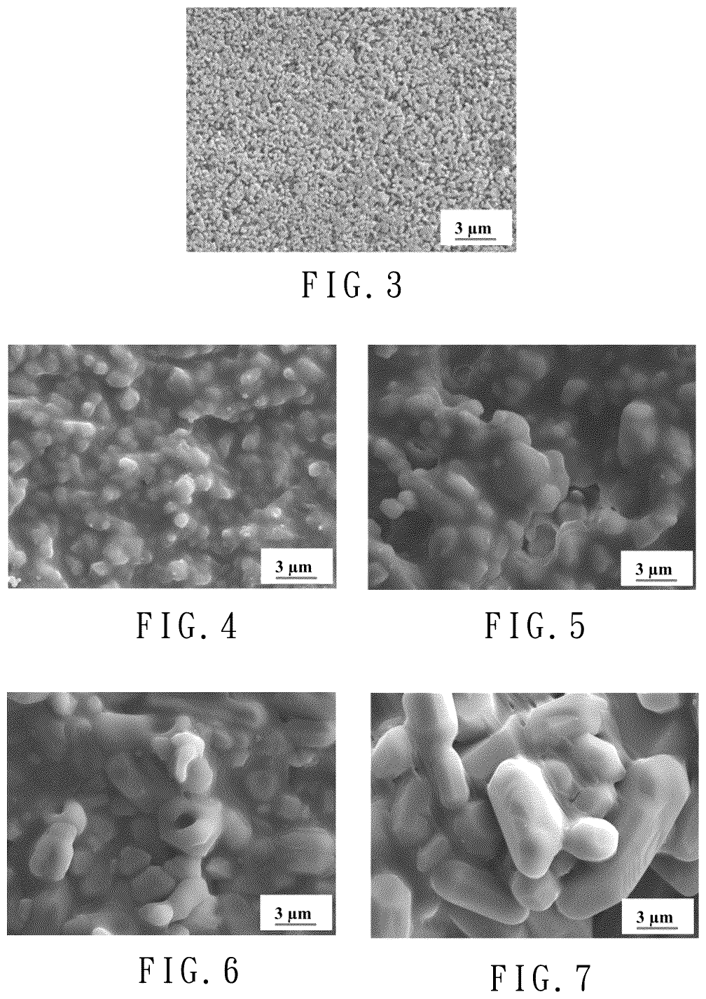

The sample was observed by scanning electron microscopy (SEM). The results are shown in FIGS. 3 to 7, pure diopside (Li0.00) as the comparative example has a uniform surface, with fine pores distributed thereon (FIG. 3). Li0.25 has a structure composed of grains embedded in a glassy matrix, showing the generation of the amorphous phase (FIG. 4). With the increase of the replacement ratio of elemental lithium, the grain aggregation and growth become more and more obvious. As a result, the porous structure increases gradually, and the grain size increases gradually (FIGS. 4-7).

Based on the crystallized phase changes and SEM images, it can be seen that the doping of elemental lithium promotes the formation of wollastonite crystallized phase, and the formation of wollastonite crystallized phase increases the porosity. The doping of elemental lithium also causes the formation of an amorphous glassy matrix. The glassy matrix promotes the improvement of the density of the ceramic. As shown in FIG. 4, the amorphous matrix of lithium disilicate is filled in the pores of the glass ceramic, so a dense surface can still be observed. In FIGS. 5-7, as the replacement ratio of elemental lithium increases, porosity caused by the formation of wollastonite and the growth of grains is greater than the densification caused by lithium disilicate, so the surface of the glass ceramic is porous.

### (IV) Promotion on Formation of HAp

To evaluate the in-vivo promotion of the glass ceramic of the present invention on the formation of hydroxyapatite (HAp) on the surface of an artificial implant, a glass ceramic specimen was immersed in a simulated body fluid (pH 7.4) at 37° C. After 28 days, the formation of HAp (Ca₅(PO₄)₃(OH)) was observed by scanning electron microscopy.

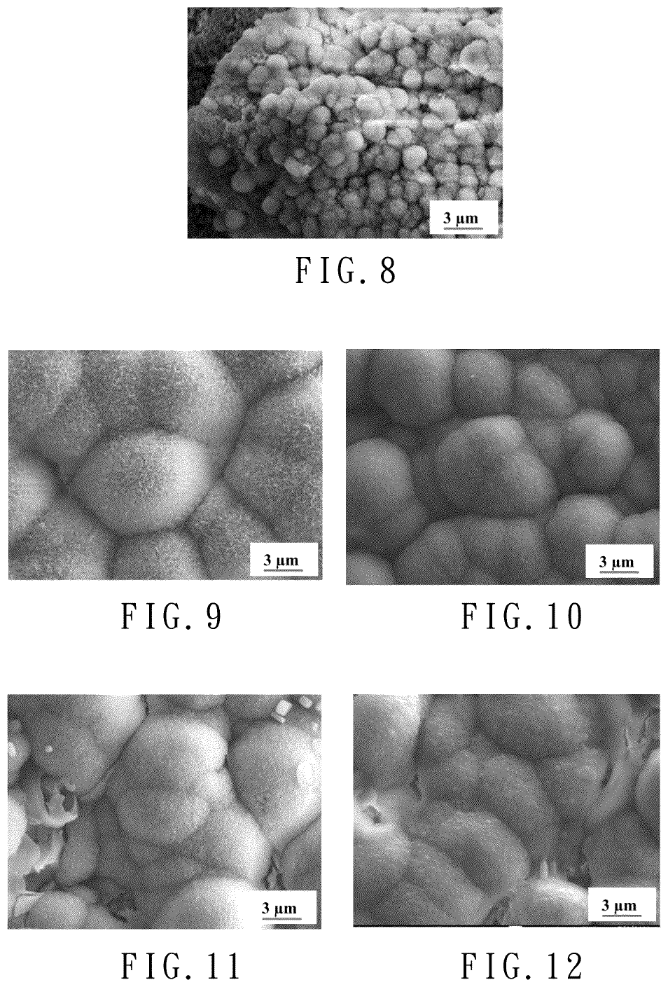

From the comparison of SEM images (FIGS. 3-7) before soaking and SEM images (FIGS. 8-12) after soaking, it can be observed that after soaking in the simulated human body fluid, spherical HAp precipitates are formed on the surface of all samples. It can be further observed that the spherical HAp precipitate of pure diopside (Li0.00) as the comparative example has a small diameter (FIG. 8), and the spherical HAp precipitate formed on the surface of the glass ceramic has a large diameter (FIGS. 9-12), showing that the doping of elemental lithium promotes the formation of HAp.

### (V) Hardness and Degradability

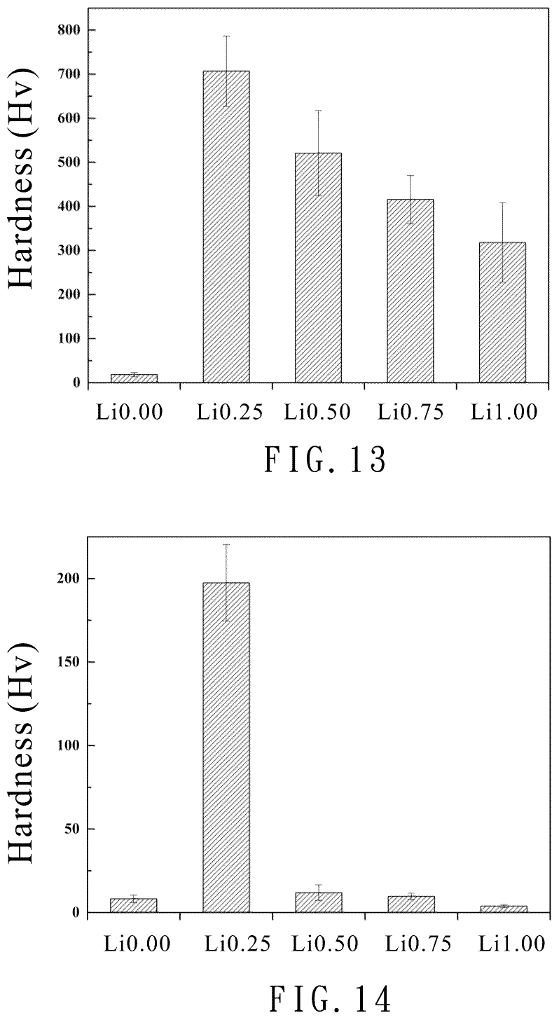

To evaluate the hardness of the glass ceramic of the present invention, the hardness was measured by a micro Vickers hardness tester. The results are shown in FIG. 13. In the test examples, the hardness of Li0.25 is the highest, which is about 38 times that of pure diopside (Li0.00). As the replacement ratio of elemental lithium increases, although the hardness of Li0.50, Li0.75 and Li1.00 glass ceramics decreases slightly, it is still higher than that of pure diopside (Li0.00).

As can be seen from the above results, elemental magnesium is replaced by a certain amount of elemental lithium (for example, 25% of elemental magnesium is replaced) in the glass ceramic of the present invention, such that the glass ceramic has a dense crystal structure, thereby improving the hardness of the glass ceramic. As the replacement ratio of elemental lithium increases, the surface of the glass ceramic becomes porous, and the hardness decreases accordingly.

In addition, to evaluate the in-vivo hardness change and degradability of the glass ceramic, the sample was immersed in a simulated human body fluid (pH 7.4) at 37° C. After 28 days, the hardness was determined, and the weight change was determined after 7, 14, 21 and 28 days.

As shown in FIGS. 13 and 14, compared with the hardness before soaking in the simulated human body fluid, the hardness of all samples decreases. The hardness of Li0.25 after soaking is the highest, showing that it has a better anti-degradation ability. Therefore, after an artificial implant made of the glass ceramic is implanted into the human body, it can maintain a certain hardness in the human body to provide sufficient support while degrading gradually and slowly.

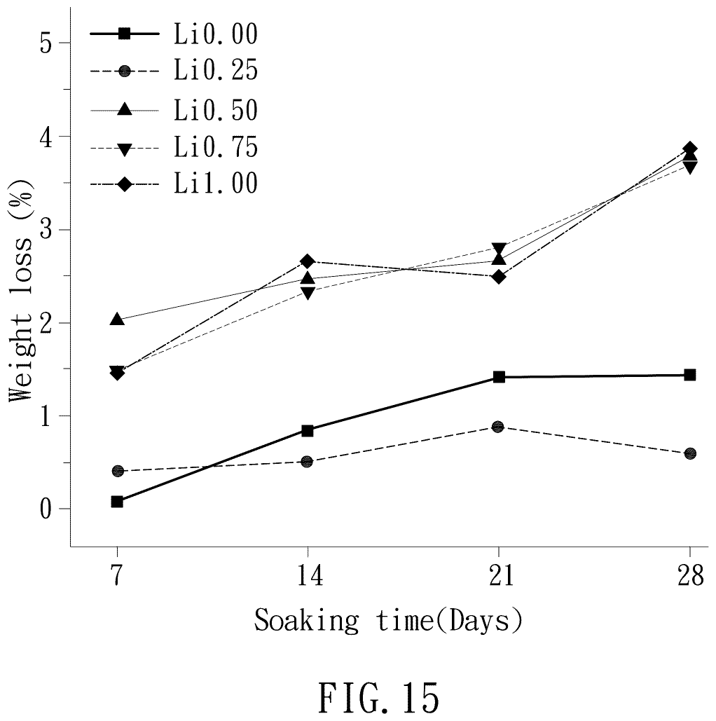

The weight changes are shown in FIG. 15. The weight loss rate of pure diopside (Li0.00) and Li0.25 is small, and the weight loss rates of Li0.50, Li0.75 and Li1.00 are high. The weight change is not only related to degradation (weight loss), but also related to the formation (weight increase) of HAp on the surface of the sample. Specifically, after the samples are immersed in simulated human body fluids (pH 7.4) at 37° C. for 28 days, the weight loss rates are all below 5%.

### (IV) Impact on pH Value of Surrounding Tissues

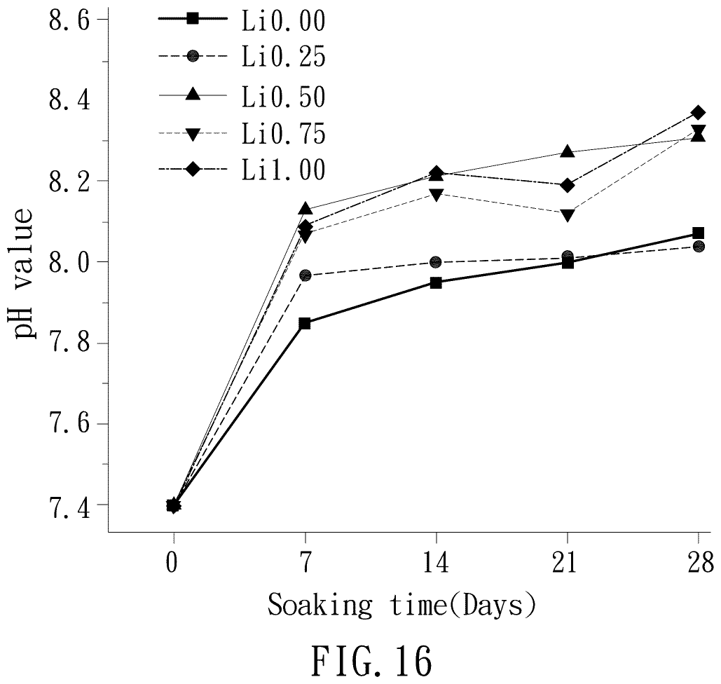

To evaluate the in-vivo impact of the glass ceramic of the present invention on the pH value of surrounding tissues, the sample was immersed in a simulated human body fluid (pH 7.4) at 37° C. The pH was measured by a PH meter at days 0, 7, 14, 21 and 28. The results are shown in FIG. 16. During the 28-day soaking, the pH values of all samples tend to rise, reaching a weakly alkaline range (between pH 8.0 and 8.5) at day 28. This environment is not only suitable for the formation of osteoblast, but also hinders the differentiation of osteoclast, thereby promoting ossification.

In summary, the glass ceramic according to the present invention is applicable to bone defect repair. For example, the glass ceramic can be prepared into an artificial implant, for implantation into the human body. Due to the specific composition of the crystallized phase of the glass ceramic, the glass ceramic has the advantages of both wollastonite-based bioceramics and diopside-based bioceramics, and has not only good hardness, but also proper degradability. During bone defect repair, an artificial implant made of the glass ceramic can provide sufficient support while degrading gradually and slowly. Compared with wollastonite-based bioceramics, the glass ceramic will not greatly reduce the pH value in an environment surrounding the artificial implant during the degradation process, thereby reducing the irritation to the surrounding environment. Compared with diopside-based bioceramics, the glass ceramic can promote the formation of HAp on the surface of the artificial implant, so as to promote the attachment of bone cells. In short, the glass ceramic has a good bone repair effect, which is a key advantage of the present invention.

Moreover, by the method for manufacturing a glass ceramic according to the present invention, a glass ceramic with good bone repair effect as described above can be manufactured, which is a beneficial effect of the present invention.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims.

What is claimed is:

1. A glass ceramic, comprising:
a major crystallized phase, which is either diopside or wollastonite; and
a minor crystallized phase, which comprises any one or more selected from the group consisting of diopside, wollastonite, lithium disilicate, silicon dioxide, lithium metasilicate and Li₂Ca₂Si₅O₁₃,
wherein in the glass ceramic, a molar ratio of elemental calcium, elemental lithium and elemental silicon is 1:x:2, in which x is 0.05 to 1;
however, when the major crystallized phase is diopside, the minor crystallized phase does not comprise diopside; and when the major crystallized phase is wollastonite, the minor crystallized phase does not comprise wollastonite.

2. The glass ceramic as claimed in claim 1, further comprising elemental magnesium, wherein a molar ratio of elemental lithium and elemental magnesium is x:(1-x), in which x is greater than or equal to 0.05 and less than 1.

3. The glass ceramic as claimed in claim 2, wherein x is 0.05 to 0.75.

4. The glass ceramic as claimed in claim 3, wherein x is 0.25.

5. A method for manufacturing a glass ceramic according to claim 1, dissolving a glass ceramic raw material to obtain a solution of the glass ceramic raw material;
adding a precipitant to the solution of the glass ceramic raw material to obtain a precipitate of the glass ceramic raw material;
calcining the precipitate of the glass ceramic raw material to obtain a glass ceramic composition, wherein in the glass ceramic composition, a molar ratio of elemental calcium, elemental lithium and elemental silicon is 1:x:2, in which x is 0.05 to 1; and
sintering the glass ceramic composition.

6. The method for manufacturing the glass ceramic as claimed in claim 5, wherein the glass ceramic raw material comprises: 17-23% by weight (wt %) of calcium chloride, 0.1-10 wt % of lithium chloride and 67-78 wt % of tetraethyl silicate.

7. The method for manufacturing the glass ceramic as claimed in claim 5, wherein the glass ceramic composition further comprises elemental magnesium, wherein a molar ratio of elemental lithium and elemental magnesium is x:(1-x), in which x is greater than or equal to 0.05 and less than 1.

8. The method for manufacturing the glass ceramic as claimed in claim 7, wherein the glass ceramic raw material comprises: 17-23% by weight (wt %) of calcium chloride, 0.1-10 wt % of lithium chloride, 67-78 wt % of tetraethyl silicate and magnesium chloride which is less than or equal to 15 wt % and greater than 0 wt %.

9. The method for manufacturing the glass ceramic as claimed in claim 5, wherein the calcining is carried out at a temperature of 400-800° C. for 2-6 hrs.

10. The method for manufacturing the glass ceramic as claimed in claim 5, wherein the sintering is carried out at a temperature of 900-1200° C. for 2-6 hrs.
